(19) United States
(12) Patent Application Publication
(10) Pub. No.: US 2008/0318289 A1
(43) Pub. Date: Dec. 25, 2008

(54) Fermentation Processes for the Preparation of Tacrolimus

(76) Inventors: Parveen Kumar, Panchkula (IN); Hitesh Kumar Malviya, Indore (IN); Raj Kumar Maurya, Ghaziabad (IN); Aniruddha Shukla, New Delhi (IN)

Correspondence Address: RANBAXY INC., 600 COLLEGE ROAD EAST, SUITE 2100, PRINCETON, NJ, 08540, US

(21) Appl. No.: 12/089,279

(22) PCT Filed: May 10, 2006
(86) PCT No.: PCT/IB06/02788

§ 371 Date: Aug. 27, 2008

(30) Foreign Application Priority Data
Oct. 5, 2005 (IN) 2673/DEL/2005

(51) Int. Cl. C12P 17/16

(52) U.S. Cl. 435/118

(57) ABSTRACT

The present invention relates to improved fermentation processes for the preparation of tacrolimus or a salt or derivative thereof by culturing a microorganism capable of producing tacrolimus or a salt or derivative thereof under submerged aerobic conditions with fed-batch processing at a high aeration rate.

### FIELD OF THE INVENTION

The present invention relates to improved fermentation processes for the preparation of tacrolimus or a salt or derivative thereof by culturing a microorganism capable of producing tacrolimus or a salt or derivative thereof under submerged aerobic conditions with fed-batch processing at a high aeration rate.

### BACKGROUND OF THE INVENTION

Tacrolimus or FK-506 of Formula I, is a macrolide antibiotic reported by Kino et al., J. Antibiotics 40, 1249-1255, 1984. The potent immunosuppressive activity of tacrolimus is described in U.S. Pat. No. 4,894,366. Tacrolimus is reportedly one hundred times more potent than the most commonly prescribed immunosuppressant, cyclosporin. Tacrolimus and other immunosuppressants such as rapamycin, cyclosporin or their combinations are useful in the prevention of graft rejection in bone marrow and organ transplants and also in the treatment of various auto-immune diseases. Tacrolimus has been shown to be effective in treating a number of diseases like asthma (PCT Application No WO 90/14826), inflammatory and hyperproliferative skin disease and cutaneous manifestations of immunologically induced illness (European Patent No 315,978).

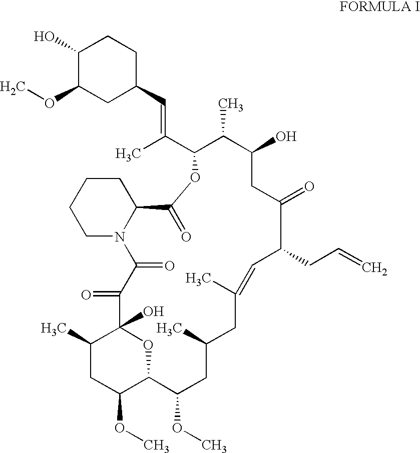

U.S. Pat. Nos. 4,894,366, 5,565,559, 5,624,842 and 5,830,717 disclose a fermentation process for production of tacrolimus using Streptomyces tsukubaensis under submerged aerobic cultural conditions.

U.S. Pat. No. 5,194,378 discloses production of tacrolimus by fermentation of Streptomyces species, ATCC No. 55098, under submerged aerobic conditions in an aqueous nutrient medium at a pH of about 7.

U.S. Pat. No. 5,324,644 discloses a process for producing the immunosuppressant agent L-682,993, also described as 31-desmethoxy-31-hydroxy-FK-506, using the new mutant microorganism Streptomyces sp. (MA 7017) ATCC No. 55334, being a blocked second generation mutant of Streptomyces sp. MA 6858. The process includes culturing the microorganism under aerobic fermentation conditions in an aqueous carbohydrate medium containing a nitrogen nutrient.

WO 04/22767 discloses a process for production of rapamycin by solid substrate fermentation of Streptomyces hygroscopicus by fed-batch technique. However solid state fermentation suffers from various disadvantages. Firstly the quality control of solid substrates like wheat bran, wheat rawa and/or oatmeal used in the process is not well standardized. Hence the use of low quality/unprocessed solid substrates as raw materials in the fermentation medium would make the recovery process more tedious due to other contaminants coming from cereal grains. The improvement in the production of rapamycin reported in the '767 application is not very significant (˜1.6 fold) so as to be suited for industrial scale preparation. Secondly the process is associated with high heterogeneity and inconsistency as it is well known that the transfer of oxygen and nutrients essential for obtaining higher productivity during commercial fermentation using a solid substrate is grossly inadequate, particularly with respect to production of high value secondary metabolites. The above difficulties make solid-state fermentation less attractive commercially due to poor scale up/modeling.

### SUMMARY OF THE INVENTION

In one general aspect there is provided a process for the manufacture of a compound of Formula I or salt or derivative thereof,

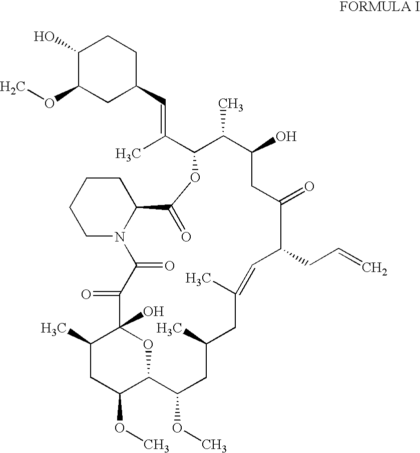

wherein the process comprises incubating a microorganism capable of producing the compound of Formula I or a salt or derivative thereof in a nutrient medium under submerged aerobic conditions with fed batch culturing at a high aeration rate.

Embodiments of the process may include one or more of the following features. For example, the high aeration rate may be 1.5 VVM or more with respect to the working volume of the bioreactor. The high aeration rate comprises 1.5-2.5 VVM with respect to the working volume of the bioreactor. The aeration rate may be 1.5 VVM with respect to the working volume of the bioreactor.

The microorganism may be a hyperproducer mutant or recombinant of Streptomyces tsukubaensis.

The process may further include providing a carbon source. The carbon source may be fed and may be fed during a production stage of fermentation. The carbon source may be selected from one or more of dextrin, starch, glucose, glycerol, xylose, galactose, glycerin, maltose, mannose, salicin, mannose, raffinose, arabinose and sodium succinate. In particular, the carbon source may be starch or dextrin.

The process may include the pH of the culture medium being between 6.5 and 7.8 and, in particular, the pH may be 7.0 to 7.5.

### DETAILED DESCRIPTION OF THE INVENTION

The prior art processes do not disclose production of tacrolimus or its salts or derivative thereof by fed-batch fermentation under submerged aerobic conditions with high aeration rates. The present inventors have found that higher production of mycelial biomass can be achieved by control of physical parameters such as aeration rate during fed batch culturing with a carbon source. This results in substantially higher production of tacrolimus in the present process wherein up to about a 5.5 fold increase in production of tacrolimus is observed as compared to batch processing or continuous processing fermentation techniques. The controlled addition of carbon source during production stage serves to control the pH range during fermentation thereby resulting in enhanced production of tacrolimus. The controlled addition also makes the present process economical. The present process is commercially viable and easily scalable at bioreactor level for producing tacrolimus on an industrial scale.

The advantages of the present process for production of tacrolimus over the prior art methods include:
(i) fed batch process under high aeration rate shows a 5.5-fold increase in tacrolimus production over batch fermentation or continuous batch process.
(ii) controlled addition of carbon substrate, which is solely required for product formation during fermentation.
(iii) under fed batch culturing, feeding of the carbon source not only supplements the nutrient medium but also enables maintenance of the desired pH range required for maximum productivity throughout fermentation.
(iv) fermentation is carried out in a contained atmosphere in a bioreactor and reduces the risk of contamination of the fermentation mixture
(v) fermentation in a bioreactor also minimizes the hazardous exposure to tacrolimus which is a potent immunosuppressant.

The term “high aeration rate” as used herein refers to an aeration rate of 1.5 VVM or more with respect to the working volume of the bioreactor. Preferably the aeration rate is 1.5-2.5 VVM with respect to the working volume of the bioreactor.

A first aspect of the present invention provides a fermentation process for the manufacture of a compound of Formula I or salt or derivative thereof,

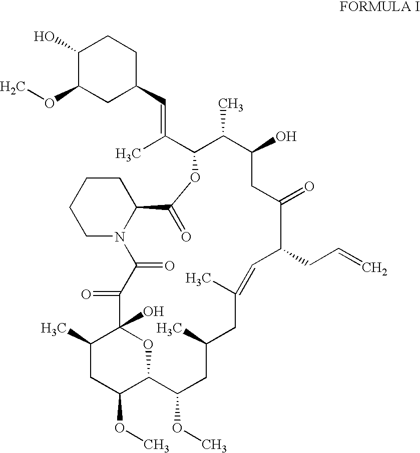

wherein the process includes incubating a microorganism capable of producing the compound of Formula I or its salts or derivatives in a nutrient medium under submerged aerobic conditions with fed batch culturing at a high aeration rate.

According to the process of the present invention tacrolimus or its salts or derivatives is produced by fermentation of a tacrolimus hyperproducer mutant strain of Streptomyces species grown in a nutrient medium containing assimilable carbon and nitrogen by fed batch culturing with a high aeration rate, preferably under submerged aerobic conditions. The aeration rate can be varied between 1.5-2.5 VVM with respect to the working volume of the bioreactor. For the production of tacrolimus in fermenters, it is preferable to employ the vegetative form of the microorganism for inoculation in order to avoid growth lag. It is desirable to produce the vegetative form of the organism first, by inoculating a relatively small quantity of the culture medium with the spores/mycelia of the organism. The cultured medium also called the “seed” is then transferred aseptically to the large fermenters. Single or double stage seed may be used for transfer to production fermenters depending upon the process/scale of operation. The fermentation medium used for the seed preparation may be the same or different from the medium utilized for the production of tacrolimus in fermenters.

The preferred sources of carbon in the nutrient medium are glucose, glycerol, starch, dextrin, xylose, galactose, glycerin, maltose, mannose, salicin, mannose, raffinose, arabinose, sodium succinate and the like. The preferred sources of nitrogen in the nutrient medium are yeast extract, peptone, cottonseed meal, soybean meal, corn steep, liquor, wheat germ, peptone, maize gluten and the like. The carbon and nitrogen sources applied in combination are quality tested and may contain traces of minerals and growth factors. Conventional fermentation agents and tracer materials may also be added. Inorganic/mineral salts such as calcium carbonate, sodium or potassium phosphate, magnesium salts and the like may also be added to the medium. If the culture medium foams excessively, antifoaming agents such as silicone oil, fatty oil, liquid paraffin or plant oil may be added.

The preferred medium for the growth and production of tacrolimus are:

(a) Seed medium containing dextrose (1 g/L), dextrin (10 g/L), cottonseed meal (3.0 g/L), yeast extract (5.0 g/L), MgSO₄.7H₂O (0.05 g/L), milk (3.0 g/L), casein enzyme hydrolysate (5.0 g/L), phosphate buffer 2.0 mL, CaCO₃ (0.5 g/L). The pH should be adjusted to 7.0±0.2 prior to sterilization.
(b) Production medium containing dextrose (0-25 g/L), dextrin (5-80 g/L), cottonseed meal (5-20 g/L), soyabean meal (5-20 g/L), soya peptone (5-20 g/L), polyethylene glycol (1-15 g/L), potassium dihydrogen phosphate (0.5-1.5 g/L), glycerol (5-20 g/L), and calcium carbonate (1-3 g/L). The pH should be adjusted to 7.0±0.2 prior to sterilization.

The production of tacrolimus or salt or derivative thereof by the present process is carried out at a pH of about 6.5 to 7.5 and a temperature of about 20° C. to 40° C. Preferably the pH is about 7.0 to 7.5 and the temperature is about 22° C. to 35° C. Preferably, the production cultures are incubated for about 70 to about 280 hours, more preferably for about 135 to 280 hours. The fermentation mixture may be agitated and aerated. Aeration may be accomplished by passage of sterile air through the fermentation mixture and agitation may be carried out mechanically or by using a propeller. The agitation of the fermentation mixture may be varied to various extents according to fermentation conditions and scale. The fermentation medium is harvested when the highest titer of tacrolimus or salt or derivative thereof is achieved. The estimation of packed mycelia volume was performed by carrying out centrifugation at 4000 RPM for 10 minutes. Tacrolimus or salt or derivative thus produced can be separated and purified from the fermentation broth by conventional methods commonly used for recovery of biologically active substances.

It is to be understood that the production of tacrolimus or salt or derivative thereof by the present process is not limited to the particular microorganism described herein, which is given for illustrative purpose only. The present invention also is intended to cover and include the use of any tacrolimus-producing mutants including natural mutants as well as artificial mutants or recombinant clones, which can be produced from tacrolimus producing microorganism or mutant thereof by conventional methods.

The specific embodiments described herein are for illustrating the present invention and should not be construed as being limitations on the scope or spirit of the present invention.

### EXAMPLE 1

### Production of Tacrolimus in Batch Culture

Step 1: A second stage seed culture was produced in a 10 Litre bioreactor by inoculating the medium containing dextrose (1.0 g/L), dextrin (10.0 g/L), cottonseed meal (3.0 g/L), yeast extract (5.0 g/L), MgSO₄.7H₂O (0.05 g/L), milk (3.0 g/L), casein enzyme hydrolysate (5.0 g/L), phosphate buffer 2.0 mL and CaCO₃ (0.5 g/L) with first stage seed obtained from shake flask culture, and incubated at 28° C. The seed culture was sufficiently grown to be used as an inoculum.
Step 2: A production medium containing dextrose (25.0 g/L), dextrin (100.0 g/L), cottonseed meal (10.0 g/L), polyethylene glycol (10.0 g/L), KH₂PO₄ (1.0 g/L), glycerol (10.0 g/L), CaCO₃ (1.0 g/L), yeast extract (10.0 g/L) and wheat peptone (2.5 g/L) was inoculated with 4-10% of the inoculum prepared in step 1. Cultivation was carried out in batch mode under aerobic conditions with aeration rate of 1 VVM at a temperature between 22° C. and 35° C. for a period of about 70 to 140 hours. The packed mycelia volume obtained during production phase was 20-25%.
Tacrolimus titre: 50-60 mg/L.

### EXAMPLE 2

### Production of Tacrolimus in Fed-Batch Culture Using Starch Feed

A second stage seed culture, as obtained in Example 1, was inoculated with 4-10% of the inoculum in a production medium containing dextrose (25.0 g/L), soluble starch (75.0 g/L), cottonseed meal (10.0 g/L), polyethylene glycol (10.0 g/L), KH₂PO₄ (1.0 g/L), glycerol, (12.5 g/L), CaCO₃ (1.0 g/L), yeast extract (10.0 g/L), corn steep liquor (10.0 g/L) and wheat peptone (2.5 g/L). Cultivation was carried out in fed batch mode under aerobic conditions with an aeration rate of 1 VVM at a temperature between 22° C. and 35° C. for a period of about 110 to 164 hours. During the course of fermentation, a 6.6% starch solution was fed and the pH was maintained in the range of 6.6-7.85, preferably in the range of 7.3-7.8. Feeding of the starch solution was started at the age of 145 hours. The packed mycelia volume obtained during production phase was 20-25%.

Tacrolimus titre: 130-150 mg/L.

### EXAMPLE 3

### Production of Tacrolimus in Fed-Batch Culture Using Dextrin Feed

A second stage seed culture, as described in Example 1 was sufficiently grown to be used as an inoculum and was transferred to a production medium containing dextrose (25.0 g/L), starch (75.0 g/L), cottonseed meal (12.5 g/L), soyabean meal (10.0 g/L), soya peptone (10.0 g/L), corn steep liquor (10.0 g/L), polyethylene glycol (10.0 g/L), KH₂PO₄ (1.0 g/L), glycerol (12.5 g/L) and CaCO₃ (1.0 g/L) was inoculated with 4-10% of the inoculum. Cultivation was carried out under aerobic conditions with aeration rate of 1 VVM at a temperature between 22° C. and 35° C. for a period of about 192 to 256 hours. During the course of fermentation, 10% dextrin solution was fed and the pH was maintained in the range of 6.6-7.3, preferably in the range of 6.6-6.8. Feeding of dextrin solution was started at the age of 136 hours. The packed mycelia volume obtained during production phase was 20-25%. Tacrolimus titre: 160-170 mg/L.

### EXAMPLE 4

### Production of Tacrolimus in Fed-Batch Culture Under Higher Aeration Rate Using Dextrin Feed

A second stage seed culture, as described in Example 1 was sufficiently grown to be used as an inoculum and was transferred to a production medium containing dextrose (25.0 g/L), dextrin (80.0 g/L), cottonseed meal (10.0 g/L), soyabean meal (10.0 g/L), soya peptone (10.0 g/L), polyethylene glycol (12.5 g/L), KH₂PO₄ (0.8 g/L), glycerol (10.0 g/L) and CaCO₃ (1.5 g/L) and was inoculated with 4-10% of the inoculum. Cultivation was carried out under highly aerobic conditions with an aeration rate of 2 VVM at a temperature between 22° C. and 35° C. for a period of about 192 to 279 hours. During the course of fermentation, 30% dextrin solution was fed and the pH was maintained in the range of 6.6-7.5, preferably in the range of 7.0-7.5. Feeding of dextrin solution was started at the age of 159 hours. The packed mycelia volume obtained during production phase was 50-60%. Tacrolimus titre: 300-310 mg/L.

While the present invention has been described in terms of its specific embodiments, certain modifications and equivalents will be apparent to those skilled in the art and are intended to be included within the scope of the present invention.

We claim:

1. A process for the manufacture of a compound of Formula I or salt or derivative thereof, 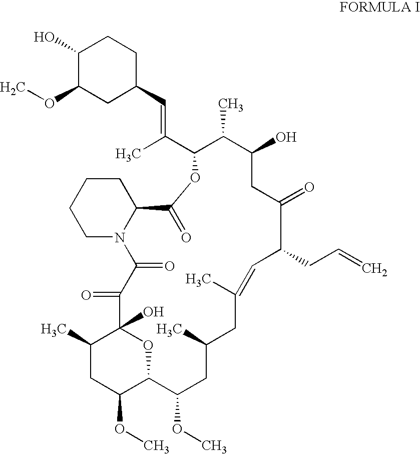
wherein the process comprises incubating a microorganism capable of producing the compound of Formula I or a salt or derivative thereof in a nutrient medium under submerged aerobic conditions with fed batch culturing at a high aeration rate.

2. The process according to claim 1 wherein the high aeration rate comprises 1.5 VVM or more with respect to the working volume of the bioreactor.

3. The process according to claim 2 wherein the high aeration rate comprises 1.5-2.5 VVM with respect to the working volume of the bioreactor.

4. The process according to claim 3 wherein the aeration rate is 1.5 VVM with respect to the working volume of the bioreactor.

5. The process according to claim 1 wherein the microorganism is a hyperproducer mutant or recombinant of Streptomyces tsukubaensis.

6. The process according to claim 1, further comprising providing a carbon source.

7. The process according to claim 6, wherein the carbon source is fed.

8. The process according to claim 7 wherein the carbon source is fed during a production stage of fermentation.

9. The process according to claim 7 wherein the carbon source is selected from one or more of dextrin, starch, glucose, glycerol, xylose, galactose, glycerin, maltose, mannose, salicin, mannose, raffinose, arabinose and sodium succinate.

10. The process according to claim 9 wherein the carbon source comprises starch or dextrin.

11. The process according to claim 1 wherein the pH of the culture medium is between 6.5 and 7.8.

12. The process according to claim 11 wherein the pH is 7.0 to 7.5.
